SEQUENCE LISTING (3 sequences)

SEQ ID NO: 1 - DNA, 20 bp, Christensenella minuta
agagtttgatcctggctcag

SEQ ID NO: 2 - DNA, 17 bp, Christensenella minuta
cggtgtgtacaagaccc

SEQ ID NO: 3 - DNA, 1261 bp, Christensenella minuta
acttcatgtgggcgggttgcagcccacaatccgaactgggaccggctttttgagattcgcttccccttacgggttcgctgccctttgtaccggccattgtagcacgtgtgtagcccaagacataaggggcatgatgatttgacgtcgtccccaccttcctccgagttgtccccggcagtctcactagagttcccgcctttacgcgctggcaactagcaataagggttgcgctcgttgcgggacttaacccaacatctcacgacacgagctgacgacaaccatgcaccacctgtctctctgccccgaagggaaactgtatctctacagtcgtcagaggatgtcaagccttggtaaggttcttcgcgttgcttcgaattaaaccacatgctccgctgcttgtgcgggcccccgtcaattcctttgagtttcaaccttgcgatcgtactccccaggcgggatacttaatgcgtttgcttcggcacggaaccctatcgggccccacacctagtatccatcgtttacggcgtggactaccagggtatctaatcctgtttgctccccacgctttcgtgcctcagtgtcagttacagtccagaaagtcgccttcgccactggtgttcctcctaatatctacgcatttcaccgctacactaggaattccacttccctctcctgtactcaagtcacacagtttcaaatgcaaccccggggttaagccccggtctttcacatctgacttacatgaccacctacgcaccctttacgcccagtaattccggacaacgcttgctccctacgtattaccgcggctgctggcacgtagttagccggagcttcctcctatggtaccgtcatttctttcgtcccataggacaaaggtttacaatccgaagaccttcttccctcacgcggcgttgctgggtcagggtttcccccattgcccaatattccccactgctgcctcccgtaggagtctggaccgtgtctcagttccagtgtggccgatcaccctctcaggtcggctacccatcgttgacttggtgggccgttacctcaccaactatctaatgggacgcgagcccatcctgcatcgaataaatccttttacctcaaaaccatgcggtttcgtggtctcatgcggtattagcagtcgtttccaactgttgtcccccgttgcagggcaggttgctcacgcgttactcacccgtccgccactcggtatacccacagttcctcccgaaggattcacaaagggcaacct

(19) United States
(12) Patent Application Publication
(10) Pub. No.: US 2022/0000942 A1
(43) Pub. Date: Jan. 6, 2022

(54) CHRISTENSENELLACEAE BACTERIA INCLUDING CHRISTENSENELLA MINUTA AND USES THEREOF

(71) Applicant: CONSEJO SUPERIOR DE INVESTIGACIONES CIENTIFICAS (CSIC), Madrid (ES)

(72) Inventors: Yolanda SANZ HERRANZ, Madrid (ES); Eva Maria GOMEZ DEL PULGAR VILLANUEVA, Madrid (ES); Ana AGUSTI FELIU, Madrid (ES); Maria Carmen CENIT LAGUNA, Madrid (ES)

(21) Appl. No.: 17/297,629

(22) PCT Filed: Nov. 27, 2019
(86) PCT No.: PCT/EP2019/082793

§ 371 Date: May 27, 2021

(30) Foreign Application Priority Data
Nov. 28, 2018 (ES) P201831153

(51) Int. Cl. A61K 35/741; C12N 1/20; A61K 35/744; A61P 25/24; A23L 33/135

(52) U.S. Cl. CPC A61K 35/741; C12N 1/205; C12R 2001/01; A61P 25/24; A23L 33/135; A61K 35/744

(57) ABSTRACT

The present invention relates to Christensenellaceae bacteria including Christensenella minuta strain DSM 32891, to the cellular components, metabolites and secreted molecules thereof, and to compositions that comprise the above products, and also to the use of said strain for the prevention and/or treatment of mood or affective disorders, such as depression, stress disorders, anxiety disorders and migraine.

### CROSS-REFERENCE TO RELATED APPLICATIONS

This application is the U.S. national stage application of PCT/EP2019/082793 with the international filing date of Nov. 27, 2019 which claims the benefit of priority from Spanish Patent Application Ser. No. P201831153, filed Nov. 28, 2018, the disclosure of which is hereby incorporated by reference in its entirety.

### SEQUENCE DISCLOSURE

This application includes, as part of its disclosure, a biological sequence listing contained in an attached text file, which is hereby incorporated by reference in its entirety.

### TECHNICAL FIELD

The present disclosure relates to Christensenellaceae bacteria including Christensenella minuta strain DSM 32891 and to the use thereof for the prevention or treatment of mood or affective disorders, such as depression, stress disorders, and anxiety disorders. The present disclosure falls within the field of the therapeutic activity of pharmaceutical compositions or preparations, and also within the field of nutrition.

### BACKGROUND OF THE INVENTION

Mood disorders and, in particular, depression, are one of the main causes of incapacity throughout the world. It is estimated that the total cost of mental disorders is 798 billion euros, of which mood disorders represent a direct and indirect annual cost of about 118 billion euros [1]. Said mood disorders include major depression, typical or melancholic and atypical depression, pre- and post-natal depression, bipolar disorder, psychotic depression, dysthymia, depressive personality disorder, seasonal affective disorder, mood disorder caused by substance abuse or by drug use, etc. In addition, the efficacy of current therapies is somewhat limited. It is estimated that only about 50% of anti-depressive treatments are effective; many patients retain sub-clinical symptomatology and others show no improvement.

Depression is a complex pathology characterized by the presence of heterogeneous symptoms, which suggests the existence of different forms of depression or phenotypes (for example, typical depression characterized by greater hyperactivity of the hypothalamic-pituitary-adrenal axis [HPA] and atypical depression characterized by greater metabolic dysregulation and an increase in appetite/weight) [2, 3]. However, little is known about the molecular mechanisms that underlie these pathologies. Depression also shows a high comorbidity with mental disorders (for example, anxiety) and physical disorders (for example, cardio-metabolic disorders, such as metabolic syndrome, diabetes and cerebrovascular disease), which adversely affect the course of the disease, reduce the therapeutic response and increase the risk of suffering from said disease.

Epidemiological research in humans has revealed associations between changes in the configuration of the gut microbiota (dysbiosis) and psychiatric disorders, such as mood or affective disorders and, among these, in particular, depression [4-9]. It is thought that these associations between dysbiosis and depression are determined to a large extent by psychosocial environmental factors (childhood trauma, work-related stress, lack of sleep) and lifestyle (unhealthy diet, sedentary lifestyle, medication) as well as by other individual characteristics such as the genome, age and sex of the person concerned, and the presence of comorbidities [2, 10]. In animal models, a stress-induced increase in the HPA axis response, a well-established risk factor for depression, causes gut dysbiosis; the dysbiotic microbiota in turn contributes to behavioral and mood disorders [11]. Animal studies also show that the specific configuration of the gut microbiota influences the response to stress, aggravating or improving the neurochemical or behavioral consequences thereof, through mechanisms that coordinate the dialog of the immune, endocrine or nervous systems [8, 12, 13].

The use of conventional probiotics (lactobacillus and bifidobacteria) for the treatment of depression has been shown to have different effects depending on the strain used. Bifidobacteria have possibly been the most commonly used to evaluate the effectiveness thereof in combating anxiety and depression; however, the results obtained have not always been conclusive or of sufficient magnitude [14, 15]

Beyond bifidobacteria, other bacterial species might be of interest for these applications, but observational studies that establish associations between different bacterial groups and depression, showing an increase or reduction in the subjects suffering from said depression, are not conclusive. Thus, for example, Yu et al. (2017) [16], observed low proportions of the bacterial groups Marvinbryantia, Corynebacterium, Psychrobacter, Christensenella, Lactobacillus, Pep-incertae sedis, Anaerovorax, Clostridialesincertae sedis and Coprococcus in mouse models of induced depression. However, Mironova et al. (2017) [17] published results showing that the microbiota of patients with Parkinson's disease and moderate depression showed a greater abundance of Christensenella minuta, Clostridium disporicum and Oscillibacter valericigenes compared with patients with Parkinson's disease and mild depression or no depression.

Accordingly, the search continues for more effective preventive and therapeutic strategies. Such strategies may improve management of mood disorders, which allows their economic and social impact to be reduced.

### DETAILED DESCRIPTION OF THE INVENTION

The present invention relates to the Christensenella minuta (C. minuta) strain DSM 32891 (C. minuta DSM 32891), to the cellular components, metabolites and secreted molecules of said strain, and to the compositions that comprise the above-mentioned products, as well as the use thereof for the prevention and/or treatment of mood disorders (such as depression), stress disorders, and anxiety disorders.

The inventors have discovered that the C. minuta strain DSM 32891 has the capacity to attenuate depressive behavior in animals exposed to acute social stress. This effect has been demonstrated by the oral administration of the bacteria (C. minuta strain DSM 32891) to an animal model of social stress which induces depressive symptoms (see example 2).

Additionally, the inventors have shown that the administration of C. minuta strain DSM 32891 modulates the hormonal stress responses in animals exposed to a 10 days social stress (example 3), indicating that diseases and conditions involving hormonal stress responses may be treatable by administration of this strain as well.

Thus, in one aspect, the present invention relates to the Christensenella minuta strain DSM 32891, hereinafter the ‘strain according to the invention,’ ‘C. minuta strain DSM 32891’ or ‘strain DSM 32891’.

The C. minuta strain DSM 32891 was isolated from the feces of healthy humans. The strain was deposited by the applicant on 7 Aug. 2018 at the Leibniz Institute DSMZ Deutsche Sammlung von Mikroorganismen und Zellkulturen GmbH (DSMZ) Inhoffenstraße 7B, D-38124 Braunschweig, Germany, as the International Depositary Authority under the Budapest Treaty—German Collection of Microorganisms and Cell Cultures. The deposit number assigned was DSM 32891. The scientific classification of the strain according to the invention is: Domain: Bacteria; Phylum: Firmicutes; Class: Clostridia; Order: Clostridiales; Family: Christensenellaceae; Species: C. minuta.

Another aspect of the invention relates to a strain derived from the C. minuta strain DSM 32891, in which said strain maintains or improves the capacities described throughout the present invention. The derived microorganism may be produced naturally or intentionally, by mutagenesis methods that are known in the prior art such as, though not limited to, the growth of the original microorganism in the presence of mutagenic or stress-producing agents, or by genetic engineering aimed at modifying specific genes. According to a preferred embodiment, the strain derived from the C. minuta strain DSM 32891 is a genetically modified mutant. The terms mutant strain or derived strain may be used interchangeably.

Another aspect of the invention relates to a bacterium comprising a 16S rRNA sequence having a specified percentage sequence identity to the 16S rRNA sequence of C. minuta strain DSM 32891 (SEQ ID NO:3), such a 16S rRNA sequence having at least 90% identity, at least 95% identity, at least 96% identity, at least 97% identity, at least 98% identity, at least 99% identity, or at least 99.5% identity to SEQ ID NO: 3. Unless otherwise indicated, such a bacterium having the aforementioned percentage identity to SEQ ID NO: 3, e.g., at least 90% identity, at least 95% identity, at least 96% identity, at least 97% identity, at least 98% identity, at least 99% identity, or at least 99.5% identity thereto, may be substituted for C. minuta strain DSM 32891 in the embodiments of the present disclosure (including the strains, methods, uses, and compositions described herein). The C. minuta strain DSM 32891 or any mutant or derivative thereof may be used in any form that produces the effects described, such as, for example, according to a preferred embodiment of the invention, the C. minuta strain DSM 32891 is in the form of viable cells (culturable or non-culturable), or according to another preferred embodiment of the invention, the strain is in the form of non-viable cells (‘dead’ cells that have been inactivated by any technique known in the prior art such as, for example, but not limited to, heat, freezing or ultraviolet radiation).

Another aspect of the present invention relates to the cellular components, metabolites, secreted molecules or any of the combinations thereof, obtained from the strain according to the invention, or from a combination of microorganisms that comprises at least one strain according to the invention.

Among the cellular components of the bacteria may be included cell wall components (such as, for example, but not limited to, peptidoglycan), nucleic acids, membrane components, or others such as proteins, lipids, carbohydrates and combinations thereof, such as lipoproteins, glycolipids or glycoproteins. Metabolites include any molecule produced or modified by the bacteria as a consequence of the metabolic activity thereof during growth, the use thereof in technological processes (for example, but not limited to, food or drug production processes), during product storage or during gastrointestinal transit. Examples of said metabolites are, but are not limited to, organic and inorganic acids, proteins, peptides, amino acids, enzymes, lipids, carbohydrates, lipoproteins, glycolipids, glycoproteins, vitamins, salts, metals or nucleic acids. Secreted molecules include any molecule exported or released outwards by the bacteria during the growth thereof, the use thereof in technological processes (for example food or drug production), product storage or gastrointestinal transit. Examples of said molecules are, but are not limited to, organic and inorganic acids, proteins, peptides, amino acids, enzymes, lipids, carbohydrates, lipoproteins, glycolipids, glycoproteins, vitamins, salts, metals or nucleic acids.

Another aspect of the present invention relates to a composition, hereinafter referred to as the ‘composition according to the invention,’ which comprises the strain according to the invention and/or the cellular components, metabolites or secreted molecules of the strain according to the invention or any of the combinations thereof.

The composition, defined generally, is a set of components which is made up of at least the strain according to the invention in any concentration; or at least of the cellular components, metabolites or secreted molecules of the strain according to the invention or any of the combinations thereof; or a combination thereof.

In a preferred embodiment, the composition according to the invention has a concentration of the strain according to the invention of between 10⁴ and 10¹⁴ colony-forming units (CFU) per gram or milliliter of the final composition. The term CFU refers to the number of bacteria able to give rise to a colony upon propagation, i.e., viable bacteria. It is to be understood that non-viable bacteria may be present in compositions as well and in general such are not expected to have any adverse effect on the properties of the live bacteria in the composition, and on the contrary they can exert an effect on their own.

In another particular embodiment, the composition according to the invention may also comprise at least one other additional microorganism that is different from the strain according to the invention and/or the cellular components, metabolites or secreted molecules thereof, or any combination thereof. For example, but not limited to, the additional microorganism which may form part of said composition is selected from among at least one of the following groups:
at least one strain of another species of the family Christensenellaceae, especially of the genus Christensenella and especially of the species Christensenella minuta;
at least one lactic bacteria or bifidobacteria of gut, alimentary or environmental origin. The lactic acid bacteria is selected from the list which comprises, but is not limited to, a bacteria of the genus Bifidobacterium, Lactobacillus, Lactococcus, Enterococcus, Propionibacterium, Leuconostoc, Weissella, Pediococcus or Streptococcus;
at least one strain of other phylogenetic groups, genera or species of prokaryotes of gut, alimentary or environmental origin, such as, for example, but not limited to, Archaea, Firmicutes, Bacteroidetes, Proteobacteria, Actinobacteria, Verrucomicrobia, Fusobacteria, Metanobacteria, Spirochaetes, Fibrobacteres, Deferribacteres, Deinococcus, Thermus, Cianobacteria, Methanobrevibacterium, Peptostreptococcus, Ruminococcus, Coprococcus, Subdolingranulum, Dorea, Bulleidia, Anaerofustis, Gemella, Roseburia, Catenibacterium, Dialister, Anaerotruncus, Staphylococcus, Micrococcus, Propionibacterium, Enterobacteriaceae, Faecalibacterium, Bacteroides, Parabacteroides, Prevotella, Eubacterium, Akkermansia, Bacillus, Butyrivibrio or Clostridium;
at least one strain of fungus or yeast such as, for example, but not limited to those belonging to the genus Saccharomyces, Candida, Pichia, Debaryomyces, Torulopsis, Aspergillus, Rhizopus, Mucor or Penicillium.

Said additional microorganism may be a strain of the same species or of a different species or taxonomic group of microorganisms from that corresponding to the strain according to the invention. The cells comprised in the composition may be viable or non-viable and may be in any phase of the state of development or growth (latent, exponential, stationary, etc.), regardless of the morphology presented. In a particular embodiment, said additional microorganism comprises at least one gut bacteria or a lactic acid bacteria.

Optionally, in another particular embodiment, the composition according to the invention may also comprise at least one bioactive component (active substance, active ingredient or therapeutic agent), such as, for example, components of foods, plant products and/or drugs.

The term ‘bioactive component’ refers to a compound having biological activity in the field of application of the patent which may improve or complement the activity of the C. minuta strain DSM 32891, including ingredients or components of foods (for example, and not limited to, polyunsaturated fatty acids, conjugated linoleic acid, prebiotics, fiber, guar gum, glucomannan, chitosan, copper picolinate, calcium, etc.), other probiotics, plants, extracts or components of plants, and drugs.

In a particular embodiment, the composition according to the invention is a pharmaceutical composition. The pharmaceutical composition is a set of components which is made up of at least the strain according to the invention in any concentration; or at least of the cellular components, metabolites or secreted molecules of the strain according to the invention or any of the combinations thereof, which has at least one application in improving the physical, physiological or psychological well-being of a subject, which results in an improvement in the general state of the health of said subject or a reduction in the risk of illness. Said pharmaceutical composition may be a medicine.

The term ‘medicine’ has a more limited meaning than that of ‘pharmaceutical composition’, as defined in the present invention, as a medicine or a medicament necessarily has a preventive or therapeutic effect. The medicine referred to which the present invention relates may be for human or veterinary use. A ‘medicine for human use’ is any substance or combination of substances which is presented as having properties for the treatment or prevention of diseases in human beings or which may be used in human beings or be administered to human beings in order to restore, correct or modify the physiological functions producing a pharmacological, immunological or metabolic action, or to establish a medical diagnosis. The term ‘medicine for veterinary use’ is any substance or combination of substances which is presented as having curative or preventive properties in relation to animal diseases or which may be administered to the animal in order to re-establish, correct or modify the physiological functions thereof producing a pharmacological, immunological or metabolic action, or to establish a veterinary diagnosis. Also considered ‘veterinary medicines’ are ‘pre-mixtures for medicated feedingstuffs’ prepared for incorporation in a feedingstuff.

In addition to the requirement for therapeutic efficacy where said pharmaceutical composition may require the use of other therapeutic agents, there may be additional fundamental reasons which for the most part require or make advisable the use of a combination of a compound according to the invention and a bioactive component, where an activity is claimed for said bioactive component such as to constitute a medicine. Said compound according to the invention obviously relates to the strain according to the invention, or to the strain derived therefrom, or to the cellular components, metabolites or secreted molecules, or any of the combinations thereof, obtained from the strain according to the invention.

In a particular embodiment, the pharmaceutical composition also comprises at least one pharmaceutically acceptable vehicle and/or excipient.

The ‘vehicle’ or carrier is preferably an inert substance. The function of the vehicle is to facilitate the incorporation of other compounds, to allow better dosage and administration or to give consistency and form to the pharmaceutical composition. Exemplary carriers include cryoprotectants or lyoprotectants. Thus, the vehicle is a substance which is used in the medicine to dilute any of the components of the pharmaceutical composition according to the present invention up to a fixed volume or weight; or alternatively that even without diluting said components, said vehicle is capable of allowing better dosage and administration or of giving consistency and form to the medicine. When the presentation form is liquid, the pharmaceutically acceptable vehicle is the diluent.

Exemplary compositions of the disclosure include a bacterium of the present disclosure, e.g., C. minuta bacteria strain DSM 32891, and a ‘lyoprotectant’, which is a substance which, when combined with a bacterium, e.g., a bacterium of the present disclosure, significantly reduces chemical and/or physical instability thereof upon dehydration (e.g., lyophilization) and/or subsequent storage. Exemplary lyoprotectants include sugars and their corresponding sugar alcohols, such as sucrose, lactose, trehalose, dextran, erythritol, arabitol, xylitol, sorbitol, and mannitol; amino acids, such as arginine or histidine; lyotropic salts, such as magnesium sulfate; polyols, such as propylene glycol, glycerol, poly(ethylene glycol), or polypropylene glycol); and combinations thereof. Additional exemplary lyoprotectants include gelatin, dextrins, modified starch, and carboxymethyl cellulose. Preferred sugar alcohols are those compounds obtained by reduction of mono- and di-saccharides, such as lactose, trehalose, maltose, lactulose, and maltulose. Additional examples of sugar alcohols are glucitol, maltitol, lactitol and isomaltulose. The lyoprotectant is generally added to the pre-lyophilized formulation in a ‘lyoprotecting amount.’ This means that, following lyophilization of the bacteria in the presence of the lyoprotecting amount of the lyoprotectant, the bacteria retain viability (ability to form colonies upon reconstitution) to a greater extent than if dehydrated and stored in the same way in the absence of the lyoprotectant.

Exemplary compositions of the disclosure include bacteria of the present disclosure, such as C. minuta bacteria strain DSM 32891, and a ‘cryoprotectant’, which is a substance used to protect said bacteria from damage during freezing and thawing. The cryoprotectant may be any additive as long as it protects the bacteria from damage during freezing and thawing. Examples of cryoprotectants include, but are not limited to, sugars (e.g. sucrose, fructose, trehalose), polyalcohols (e.g. glycerol, sorbitol, mannitol), polysaccharides (e.g. celluloses, starch, gums, maltodextrin), polyethers (e.g. polypropylene glycol, polyethylene glycol, polybutylene glycol), antioxidants (e.g. natural antioxidants such as ascorbic acid, beta-carotene, vitamin E, glutathione, chemical antioxidants), oils (e.g. rapeseed oil, sunflower oil, olive oil), surfactants (e.g. Tween20, Tween80, fatty acids), peptones (e.g. soy peptones, wheat peptone, whey peptone), tryptones, vitamins, minerals (e.g. iron, manganese, zinc), hydrolysates (e.g. protein hydrolysates such as whey powder, malt extract, soy), amino acids, peptides, proteins, nucleic acids, nucleotides, nucleobases (e.g. cytosine, guanine, adenine, thymine, uracil, xanthine, hypoxanthine, inosine), yeast extracts (e.g. yeast extracts of Saccharomyces spp., Kluyvermomycesa spp., or Torula spp.), beef extract, growth factors, and lipids.

The term ‘excipient’ refers to a substance that helps the absorption of any of the components of the composition according to the present invention, stabilizes said components or helps the preparation of the pharmaceutical composition in the sense of giving said pharmaceutical composition consistency or adding flavors that render said pharmaceutical composition more agreeable. Thus, the excipients may have a function of keeping the components together such as, for example, starches, sugars or celluloses, a sweetening function, a colorant function, a protective function of the medicine such as, for example, isolating said medicine from air and/or moisture, a filler function for a pill, capsule or any other presentation form such as, for example, dibasic calcium phosphate, a disintegrant function to facilitate the dissolution of the components and the absorption thereof in the gut, without excluding other types of excipients not mentioned in this paragraph. Thus, the term ‘excipient’ is defined as that material which, included in the galenic forms, is added to the active ingredients or to the associations thereof to make possible the preparation and stability thereof, to modify the organoleptic properties thereof or to determine the physicochemical properties of the pharmaceutical composition and the bioavailability thereof. The ‘pharmaceutically acceptable’ excipient must allow the activity of the compounds of the pharmaceutical composition, that is, must be compatible with said components.

In addition, as will be understood by a person skilled in the art, the excipient and the vehicle, when present, must be pharmacologically acceptable, that is, the excipient and the vehicle must be permitted and evaluated so as not to cause injury to the organisms to which said excipients or vehicles are administered.

The pharmaceutical composition or medicine may be presented in any clinically permitted administration form and in a therapeutically effective amount. For example, said pharmaceutical composition or medicine may be in a form suitable for oral, sublingual, nasal, intrathecal, bronchial, rectal, transdermal, inhaled or parenteral administration, preferably in a form suitable for oral administration. The pharmaceutical composition according to the invention may be formulated in solid, semi-solid, liquid or gaseous forms, such as a tablet, capsule, powder, pellet, ointment, solution, suppository, injection, inhalant, gel, microbead or aerosol. The form suitable for oral administration is selected from the list which comprises, but is not limited to, drops, syrup, tisane, elixir, suspension, extemporaneous suspension, drinkable vial, tablet, capsule, pellet, cachet, pill, tablet, pastille, lozenge, or in freeze dried form. In a particular embodiment, the composition according to the invention is presented in a form suitable for oral, sublingual, nasal, bronchial, lymphatic, rectal, transdermal, inhaled or parenteral administration.

In a more particular embodiment, the composition according to the invention is presented in a form suitable for oral administration. The form suitable for oral administration refers to a physical state which may allow oral administration. Said form suitable for oral administration is selected from the list which comprises, but is not limited to, drops, syrup, tisane, elixir, suspension, extemporaneous suspension, drinkable vial, tablet, capsule, pellet, cachet, pill, caplet, pastille, lozenge, or in freeze dried form.

The ‘galenic form’ or ‘pharmaceutical form’ is the arrangement by which the active ingredients and excipients are adapted to form a medicine. It is defined as the combination of the form in which the pharmaceutical composition is presented by the manufacturer and the form in which said pharmaceutical composition is administered. In the present invention, the expression ‘effective amount’ or ‘therapeutically effective amount’ refers to that amount of the component of the pharmaceutical composition which when administered to a mammal, preferably a human, is sufficient to produce the prevention and/or treatment, as defined below, of an illness or pathological condition of interest in the mammal, preferably a human. The therapeutically effective amount will vary, for example, according to the activity of the strain according to the invention; of the cellular components, metabolites, secreted molecules or any of the combinations thereof, in any presentation form; the therapeutically effective amount will also vary according to the metabolic stability and duration of the action of the compound; the age, body weight, general state of health, sex and diet of the patient; the manner and time of administration, the speed of excretion, the combination of drugs; the severity of the particular disorder or pathological condition; and the subject who undergoes therapy, but may be determined by a person skilled in the art according to his or her own knowledge and this description.

As an alternative to the pharmaceutical composition, the composition according to the invention may also be a nutritional composition.

The term ‘nutritional composition’ according to the present invention refers to that foodstuff which, while providing nutrients to the subject who takes said nutritional composition, beneficially affects one or more functions of the organism, so as to provide a better state of health and well-being. Consequently, said nutritional composition may be intended for the prevention and/or treatment of an illness or of the factor causing an illness. Thus, the term ‘nutritional composition’ according to the present invention may be used as a synonym for a functional foodstuff or a foodstuff for specific nutritional purposes or a medicinal foodstuff.

In a particular embodiment, the nutritional composition is a foodstuff, a supplement, a nutraceutical, a probiotic or a symbiotic.

In a more particular embodiment, the foodstuff is selected from the list which comprises a milk product, plant product, meat product, aperitif, chocolate, drink or baby food. The milk product is selected from the list which comprises, but is not limited to, a product derived from fermented milk (for example, but not limited to, yogurt or cheese) or unfermented milk (for example, but not limited to, ice cream, butter, margarine, whey). The plant product is, for example, but is not limited to, a cereal in any presentation form, whether fermented or not fermented. The drink may be, but is not limited to, any fruit juice or unfermented milk.

The term ‘supplement,’ a synonym for any of the terms ‘dietary supplement,’ ‘nutritional supplement’ or ‘food supplement’ is a ‘food ingredient’ intended to complement nutrition. Some examples of dietary supplements are, but are not limited to, vitamins, minerals, botanical products, amino acids and components of foods such as enzymes and glandular extracts. Said supplements are not presented as substitutes for a conventional foodstuff or as a single component of a meal or of a nutritional diet, but as a dietary complement.

The term ‘nutraceutical’ as used in the present invention refers to substances that have been isolated from a foodstuff and used in a dosed form which have a beneficial effect on health.

The term ‘probiotic’ as used in the present invention refers to living microorganisms which when supplied in suitable amounts promote benefits in the health of the host organism.

The term ‘symbiotic’ as used in the present invention refers to those foodstuffs that contain a mixture of prebiotics and probiotics. As a general rule, said foodstuffs contain a prebiotic component which promotes the growth and/or the metabolic activity and ultimately the effect of the probiotic with which said foodstuff is combined, such as, for example, but not limited to, the possible association of fructooligosaccharides or galactooligosaccharides with bifidobacteria.

Another aspect of the present invention refers to the use of the strain according to the invention, or the components derived therefrom, or the composition according to the invention, for the manufacture of a medicine, of a nutritional composition or of a foodstuff.

Another aspect of the present invention relates to the C. minuta strain DSM 32891, a cellular component, metabolite, secreted molecule or any of the combinations thereof obtained from the strain according to the invention, or the composition according to the invention, for use as a medicine. The term medicine has been defined previously, and applies to the present aspect of the invention.

In another aspect, the present invention relates to the strain according to the invention, a strain derived therefrom, a cellular component, metabolite, secreted molecule or any of the combinations thereof obtained from the strain according to the invention, or the composition according to the invention, for use in the prevention and/or treatment of mood disorders (such as depression), stress disorders, and anxiety disorders.

In the present invention, the term ‘treatment’ refers to combatting the effects caused as a consequence of an illness or pathological condition of interest in a subject (preferably a mammal, and more preferably, a human) which includes:

(i) inhibiting the illness or pathological condition, that is, slowing the development thereof;
(ii) alleviating the illness or pathological condition, that is, causing the regression of the illness or pathological condition or of the symptomatology thereof;
(iii) stabilizing the illness or pathological condition.

In the present invention, the term ‘prevention’ refers to preventing the appearance of the illness, that is, preventing the illness or pathological condition being produced in a subject (preferably a mammal, and more preferably a human), in particular, when said subject has a predisposition to the pathological condition.

In the present invention, mood disorders or disturbances include, but are not limited to, depression, major depression, atypical depression, typical or melancholic depression, psychotic depression, catatonic depression, pre- and post-natal depression, bipolar disorder, seasonal affective disorder, dysthymia, depressive personality disorder, double depression, unspecified depressive disorder, recurrent brief depressive disorder, minor depression, mood disorder induced by substance abuse or by the use of drugs, etc.

Exemplary methods of the present disclosure provide for the prevention or treatment of disorders associated with trauma and stressor-related disorders, in which exposure to a traumatic or stressful event is listed explicitly as a diagnostic criterion. Such disorders are referred to herein as “stress disorders” and include, for example, reactive attachment disorder, disinhibited social engagement disorder, posttraumatic stress disorder (PTSD), acute stress disorder, and adjustment disorders.

Methods of the present disclosure also provide for the prevention or treatment of anxiety disorders, such as panic attack, panic disorder, agoraphobia, social phobia or social anxiety disorder, obsessive-compulsive disorder, post-traumatic stress disorder (ASD), generalized anxiety disorder, or acute stress disorder.

Without intent to be limited by theory, it is believed that the aforementioned stress disorders and anxiety disorders are characterized by alteration in the levels of epinephrine, norepinephrine, serotonin, and/or dopamine, such that the modulatory effects of the inventive C. minuta strain DSM 32891 on these hormones is expected to treat, ameliorate, or lessen these conditions or one or more of the symptoms thereof. Subjects amenable to treatment according to the invention include mammalian subjects, including humans, suffering from or at risk for any of a variety of disorders disclosed herein, including mood disorders (such as depressive disorders), anxiety disorders, and stress disorders. Within the methods of the invention, C. minuta strain DSM 32891, or a composition comprising C. minuta strain DSM 32891, is administered in an amount effective to treat a specified disorder alone or in combination with a psychotherapeutic drug including, but not limited to, drugs from the general classes of anti-depressant, mood-stabilizing, anxiolytic, anticonvulsant, antipsychotic, antiaddictive, appetite suppressant drugs and opiate agonists. (See, e.g., R J. Baldessarini in Goodman & Gilman's The Pharmacological Basis of Therapeutics, 9th Edition, Chapter 18, McGraw-Hill, 1996).

The methods of the present disclosure may be used for the treatment or prevention of disorders in human or non-human animals. Exemplary non-human animals include, without limitation, companion animals, livestock animals, zoo animals, mammals, etc., such as dogs, cats, guinea pigs, ferrets, hamsters, pigs, cows, goats, sheep, horses, mice, rats, etc.

Said treatment or prevention of disorders may comprise the administration of an effective amount of C. minuta strain DSM 32891. The amount of bacteria administered may comprise between 10⁴ and 10¹⁴ colony-forming units (CFU). An effective amount may be determined based on factors known to the person skilled in the art, including without limitation thereto the age, body weight, gender, and type and severity of disorder. If said bacteria are administered in a composition, said composition has a concentration of the bacterium of between 10⁴ and 10¹⁴ colony-forming units (CFU) per gram or milliliter of final composition.

In another aspect, the present invention relates to the strain according to the invention, the strain derived from the strain according to the invention, the cellular component, metabolite, secreted molecule or any of the combinations thereof obtained from the strain according to the invention, or the composition according to the invention, for use as an adjuvant in the treatment or prevention of mood disorders (such as depression), stress disorders, and anxiety disorders.

In exemplary embodiments, the bacterium or composition thereof for use may be for administration in any clinically permitted administration form and in a therapeutically effective amount. For example, said bacterium or composition thereof may be for oral, sublingual, nasal, intrathecal, bronchial, rectal, transdermal, inhaled or parenteral administration, preferably oral administration. The pharmaceutical composition according to the invention may be formulated in solid, semi-solid, liquid or gaseous forms, such as a tablet, capsule, powder, pellet, ointment, solution, suppository, injection, inhalant, gel, microbead or aerosol. The form suitable for oral administration is selected from the list which comprises, but is not limited to, drops, syrup, tisane, elixir, suspension, extemporaneous suspension, drinkable vial, tablet, capsule, pellet, cachet, pill, tablet, pastille, lozenge, or in freeze dried form. In a particular embodiment, the composition according to the invention is presented in a form suitable for oral, sublingual, nasal, bronchial, lymphatic, rectal, transdermal, inhaled or parenteral administration, and/or is administered by such route of administration. Preferred routes of administration deliver the C. minuta strain DSM 32891 or composition thereof to the gastrointestinal tract, e.g., by oral, sublingual, or rectal administration.

In a more particular embodiment, the composition according to the invention is presented in a form suitable for oral administration, and/or is administered orally. The form suitable for oral administration refers to a physical state which may allow oral administration. Said form suitable for oral administration is selected from the list which comprises, but is not limited to, drops, syrup, tisane, elixir, suspension, extemporaneous suspension, drinkable vial, tablet, capsule, pellet, cachet, pill, caplet, pastille, lozenge, or in freeze dried form.

In the present invention ‘adjuvant’ is understood to be that compound which helps improve the effectiveness or efficiency of other medicines for the treatment of mood disorders (such as depression), stress disorders, and anxiety disorders, and migraines which would allow the dose and/or frequency of administration thereof to be reduced or increase the efficacy thereof by the administration of a formulation of the strain according to the invention with mechanisms having complementary action.

In another aspect, the present invention relates to the use of the strain according to the invention, a strain derived from the strain according to the invention, the cellular component, metabolite, secreted molecule or any of the combinations thereof obtained from the strain according to the invention, or the composition according to the invention, for the preparation of a foodstuff. The term medicine has been defined previously in the present description and applies to the present aspect of the invention. In an exemplary embodiment, the disclosure provides an isolated bacterium of the strain of Christensenella minuta with the deposit number DSM 32891.

In an exemplary embodiment, the disclosure provides an isolated bacterium of a strain derived from the strain of Christensenella minuta with the deposit number DSM 32891. In an exemplary embodiment, disclosure provides an isolated bacterium comprising a genetically modified mutant of the strain of Christensenella minuta with the deposit number DSM 32891 or a strain derived therefrom.

Said isolated bacterium may be viable or non-viable.

In an exemplary embodiment, disclosure provides cellular component, metabolite, secreted molecule or any combination thereof obtained from the bacteria described herein.

In an exemplary embodiment, disclosure provides a composition which comprises a bacterium of the strain of Christensenella minuta with the deposit number DSM 32891. Said composition may also comprise at least one bioactive component. Said composition may comprise at least one microorganism that is of a different strain than the bacterium, which may be a gut bacterium or a lactic bacterium. Said composition may be a pharmaceutical composition. Said composition may further comprise at least one pharmaceutically acceptable vehicle and/or excipient. Said composition may be presented in a form suitable for oral, sublingual, nasal, bronchial, lymphatic, rectal, transdermal, inhaled or parenteral administration. Said composition may be a nutritional composition. Said composition may be a foodstuff, a supplement, a nutraceutical, a probiotic or a synbiotic. Said foodstuff may be selected from the list that is made up of a milk product, plant product, meat product, aperitif, chocolate, drink or baby food. Said composition may have a concentration of the bacterium of between 10⁴ and 10¹⁴ colony-forming units (CFU) per gram or milliliter of final composition.

Said bacteria may be freeze-dried, wherein said composition optionally comprises a lyoprotectant, such as a sugar, sugar alcohol, amino acid, or polyol. Said lyoprotectant may comprise a sugar or sugar alcohol such as sucrose, lactose, trehalose, dextran, erythritol, arabitol, xylitol, sorbitol, mannitol, lactulose, maltulose, glucitol, maltitol, lactitol or isomaltulose; an amino acid, such as arginine or histidine; a lyotropic salt, such as magnesium sulfate; a polyol, such as propylene glycol, glycerol, poly(ethylene glycol), or polypropylene glycol); or a gelatin, dextrins, dextran, modified starch, carboxymethyl cellulose, or hydroxypropyl-beta-cyclodextrin, or a combination thereof.

Said pharmaceutically acceptable vehicle and/or excipient may comprise a cryoprotectant such as glycerol, or a cryoprotectant selected from sugars such as sucrose, fructose, trehalose; sugar alcohols, such as glycerol, sorbitol, mannitol; polysaccharides, such as celluloses, starch, gums, maltodextrin; polyethers such as polypropylene glycol, polyethylene glycol, polybutylene glycol; antioxidants such as natural antioxidants such as ascorbic acid, beta-carotene, vitamin E, glutathione, chemical antioxidants; oils such as rapeseed oil, sunflower oil, olive oil; surfactants such as Tween20, Tween80, fatty acids; peptones such as soy peptones, wheat peptone, whey peptone; tryptones, vitamins, minerals such as iron, manganese, zinc; hydrolysates such as protein hydrolysates such as whey powder, malt extract, soy; amino acids, peptides, proteins, nucleic acids, nucleotides, nucleobases such as cytosine, guanine, adenine, thymine, uracil, xanthine, hypoxanthine, inosine; yeast extracts; beef extract; growth factors; lipids; and combinations thereof.

In an exemplary embodiment, the disclosure provides a bacterium or a composition as disclosed herein for use as a medicament.

In an exemplary embodiment, the disclosure provides a therapeutic method comprising administering an isolated bacterium or a composition as disclosed herein to a subject in need thereof.

In an exemplary embodiment, the disclosure provides an isolated bacterium or a composition as disclosed herein for use in the prevention and/or the treatment of mood disorders.

In an exemplary embodiment, the disclosure provides a method of prevention and/or the treatment of mood disorders comprising administering an effective amount of an isolated bacterium or a composition as disclosed herein.

In an exemplary embodiment, the disclosure provides an isolated bacterium or a composition as disclosed herein for its use as an adjuvant in the treatments of alterations of the state of mind.

A method of treating and/or preventing an altered state of mind, comprising administering an effective amount of an isolated bacterium or a composition as disclosed herein.

The mood disorder may be selected from the list comprising: depression, major depression, atypical depression, typical or melancholic depression, psychotic depression, catatonic depression, pre- and post-partum depression, bipolar disorder, seasonal affective disorder, dysthymia, depressive personality disorder, double depression, unspecified depressive disorder, recurrent brief depressive disorder, minor depression, alterations of the state of mind and mood disorder induced by substance abuse or by the use of drugs such as drugs of abuse.

In an exemplary embodiment, the disclosure provides the use of an isolated bacterium or a composition as disclosed herein for the preparation of a food.

In an exemplary embodiment, the disclosure provides a method of making a food, comprising admixing an isolated bacterium or a composition as disclosed herein.

In an exemplary embodiment, the disclosure provides an isolated bacterium or a composition as disclosed herein for use in the prevention and/or the treatment of a stress disorder.

In an exemplary embodiment, the disclosure provides a method of treating or preventing a stress disorder, comprising administering an effective amount of an isolated bacterium or a composition as disclosed herein.

Said stress disorder may be selected from reactive attachment disorder, disinhibited social engagement disorder, posttraumatic stress disorder (PTSD), acute stress disorder, and adjustment disorders.

In an exemplary embodiment, the disclosure provides an isolated bacterium or a composition as disclosed herein for use in the prevention and/or the treatment of an anxiety disorder.

In an exemplary embodiment, the disclosure provides a method of treating or preventing an anxiety disorder, comprising administering an effective amount of an isolated bacterium or a composition as disclosed herein.

Said anxiety disorder may be selected from panic attack, panic disorder, agoraphobia, social phobia or social anxiety disorder, obsessive-compulsive disorder, post-traumatic stress disorder (ASD), generalized anxiety disorder, or acute stress disorder.

In an exemplary embodiment, the disclosure provides an isolated bacterium or a composition as disclosed herein for use as a medicine.

In an exemplary embodiment, the disclosure provides a method of treating or preventing a disease or disorder in a subject in need thereof, comprising administering an effective amount of an isolated bacterium or a composition as disclosed herein.

In an exemplary embodiment, the disclosure provides a Christensenellaceae bacterium, especially an isolated Christensenellaceae bacterium, or a composition comprising a Christensenellaceae bacterium, for use in the prevention and/or treatment of a mood disorder, stress disorder, anxiety disorder, and/or migraine.

In an exemplary embodiment, the disclosure provides a method of treating or preventing a mood disorder, stress disorder, anxiety disorder and/or migraine, comprising administering an effective amount of a bacterium or a composition as disclosed herein.

In an exemplary embodiment, the disclosure provides a Christensenellaceae bacterium or a composition comprising a Christensenellaceae bacterium, for use as an adjuvant in treatments for mood disorders and/or stress disorders and/or anxiety disorders and/or migraines.

In an exemplary embodiment, the disclosure provides a method of treating or preventing a mood disorders and/or stress and/or anxiety, comprising administering an effective amount of an isolated bacterium or a composition as disclosed herein.

The mood disorder may be selected from the list which comprises: depression, major depression, atypical depression, typical or melancholic depression, psychotic depression, catatonic depression, pre- and post-partum depression, bipolar disorder, seasonal affective disorder, dysthymia, depressive personality disorder, double depression, unspecified depressive disorder, recurrent brief depressive disorder, minor depression, alterations of the state of mind and mood disorder induced by substance abuse or by the use of drugs, such as drugs of abuse.

In the methods and uses disclosed herein, said Christensenellaceae bacteria may be bacteria of the genus Christensenella. Said Christensenella bacteria may be selected from the species Christensenella minuta, Christensenella timonensis, Christensenella massiliensis or any combination thereof. Said Christensenella bacteria may be a Christensenella massiliensis strain with the deposit number DSM 102344 or a Christensenella timonensis strain with the deposit number DSM 102800 any combination thereof. Said Christensenella bacteria may be of Christensenella minuta strain DSM 32891 or a derivative or mutant thereof, or Christensenella minuta with the deposit number DSM 22607 or a derivative or mutant thereof, or any combination thereof.

In the methods and uses disclosed herein, said Christensenellaceae bacteria may be in the form of viable cells and/or in the form of non-viable cells.

In the methods and uses disclosed herein, said Christensenellaceae bacterium may comprise a 16S rRNA sequence having at least 90% identity, at least 95% identity, at least 96% identity, at least 97% identity, at least 98% identity, at least 99% identity, or at least 99.5% identity to the 16S rRNA sequence of the strain of Christensenella minuta with the deposit number DSM 32891 (SEQ ID NO: 3), or may comprise the strain of Christensenella minuta with the deposit number DSM 32891.

The methods and uses disclosed herein may be for use in a subject selected from a human or a non-human animal. Exemplary non-human animals include companion animals, livestock animals, zoo animals, mammals, dogs, cats, guinea pigs, ferrets, hamsters, pigs, cows, goats, sheep, horses, mice, and rats.

In the methods and uses disclosed herein may comprise the administration of between 10⁴ and 10¹⁴ colony-forming units (CFU) of said Christensenellaceae bacteria, especially, if said Christensenellaceae bacteria are administered in a composition, said composition has a concentration of the bacterium of between 10⁴ and 10¹⁴ colony-forming units (CFU) per gram or milliliter of final composition.

Throughout the description and the claims, the word ‘comprises’ and variants thereof are not intended to exclude other technical characteristics, additives, components or steps. For persons skilled in the art, other objects, advantages and characteristics of the invention will become clear in part from the description and in part from the practice of the invention. The following examples and figures are supplied as an illustration and are not intended to limit the present invention.

### BRIEF DESCRIPTION OF THE SEVERAL VIEWS OF THE DRAWINGS

FIG. 1: Evaluation of the effect of the administration of B. breve MF217 (1×10⁹ CFU/day) and C. minuta DSM 32891 (1×10⁹ CFU/day) in C57BL/6 mice (n=10/group) exposed to acute social stress in the sucrose preference test (SPF). The data are expressed in grams with averages and standard error of the mean. Statistically significant differences were established by applying ANOVA for a factor followed by the post hoc Bonferroni test (*=p<0.05, **=p<0.01). C, control mice; S, mice subjected to acute stress with no treatment; S+B. bre, mice subjected to acute stress and treated with B. breve; S+C. min, mice subjected to acute stress and treated with C. minuta.

FIG. 2: Evaluation of the effect of the administration of the B. breve strain MF217 (1×10⁹ CFU/day) and C. minuta DSM 32891 (1×10⁹ CFU/day) in C57BL/6 mice (n=10/group) exposed to acute social stress in the tail suspension test (TST). The data are expressed as averages of the time (seconds) and its standard error of the mean. Statistically significant differences were established by applying ANOVA for a factor followed by the post hoc Bonferroni test (***=p<0.001). C, control mice; S, mice subjected to acute social stress with no treatment; S+B. bre, mice subjected to acute stress and treated with B. breve; S+C. min, mice subjected to acute stress and treated with C. minuta.

FIG. 3A-3D: Evaluation of the effect of administration of C. minuta DSM 32891 (1×10⁹ cfu/day) in C57BL/6 mice exposed to social stress on stress markers in plasma. The data are expressed in nM with means and standard error. The statistically significant differences were established by applying ANOVA of one factor followed by the Bonferroni post hoc test (*=p<0.05, **=p<0.001, ***=p<0.001). C, control mice; S, mice subjected to stress, untreated; S+M, mice subjected to stress and treated with C. minuta. Stress markers were measured 1 day before social defeat, 1 day after beginning of social defeat and 10 days after beginning of social defeat. Results are shown for adrenaline (FIG. 3A), noradrenaline (FIG. 3B), serotonin (FIG. 3C), and dopamine (FIG. 3D).

### EXAMPLES

Next, the invention will be illustrated by means of some tests that demonstrate the properties and effectiveness of exemplary products according to the invention. These examples are intended to illustrate, rather than limit, the invention, which is limited only by the claims.

### Example 1. Isolation and Identification of the Species of Christensenella

The biological material that was the object of the patent was isolated from feces from healthy volunteers, which was processed and inoculated into gut microbiota medium (GMM), the composition of which is based on the mediums recommended in previous publications [18],[19], with modifications designed by the inventors. Said modifications consisted of fermenting the processed feces, while maintaining a constant pH, in an anaerobic chamber, for 24 hours. The fermented GMM medium was used as a supplement for the fastidious anaerobe agar (FAA) medium with 0.5% defibrinated blood, which was used to inoculate serial dilutions of the feces and isolate colonies, after incubating the plates for 72 hours at 37° C. in an anaerobic chamber. Christensenella minuta DSM 32891 was isolated from the colonies that grew.

Identification was carried out by sequencing the 16S rRNA gene (1.26 Kb) using the 27f (SEQ ID NO: 1: 5′-AGAGTTTGATCCTGGCTCAG-3′) and 1401r (SEQ ID NO: 2: 5′-CGGTGTGTACAAGACCC-3′) primers by Sanger sequencing technology in an ABI 3730XL sequencer. By comparing the sequences obtained with those in the NCBI database and the BLASTn algorithm, the isolated strain DSM 32891 was identified with the species Christensenella minuta with a 100% identity percentage. The complete sequence (SEQ ID NO: 3) is shown below:

>16S rRNA gene sequence of Christensenella minuta
DSM 32891
ACTTCATGTGGGCGGGTTGCAGCCCACAATCCGAACTGGGACCGGCTTT
TTGAGATTCGCTTCCCCTTACGGGTTCGCTGCCCTTTGTACCGGCCATT
GTAGCACGTGTGTAGCCCAAGACATAAGGGGCATGATGATTTGACGTCG
TCCCCACCTTCCTCCGAGTTGTCCCCGGCAGTCTCACTAGAGTTCCCGC
CTTTACGCGCTGGCAACTAGCAATAAGGGTTGCGCTCGTTGCGGGACTT
AACCCAACATCTCACGACACGAGCTGACGACAACCATGCACCACCTGTC
TCTCTGCCCCGAAGGGAAACTGTATCTCTACAGTCGTCAGAGGATGTCA
AGCCTTGGTAAGGTTCTTCGCGTTGCTTCGAATTAAACCACATGCTCCG
CTGCTTGTGCGGGCCCCCGTCAATTCCTTTGAGTTTCAACCTTGCGATC
GTACTCCCCAGGCGGGATACTTAATGCGTTTGCTTCGGCACGGAACCCT
ATCGGGCCCCACACCTAGTATCCATCGTTTACGGCGTGGACTACCAGGG
TATCTAATCCTGTTTGCTCCCCACGCTTTCGTGCCTCAGTGTCAGTTAC
AGTCCAGAAAGTCGCCTTCGCCACTGGTGTTCCTCCTAATATCTACGCA
TTTCACCGCTACACTAGGAATTCCACTTCCCTCTCCTGTACTCAAGTCA
CACAGTTTCAAATGCAACCCCGGGGTTAAGCCCCGGTCTTTCACATCTG
ACTTACATGACCACCTACGCACCCTTTACGCCCAGTAATTCCGGACAAC
GCTTGCTCCCTACGTATTACCGCGGCTGCTGGCACGTAGTTAGCCGGAG
CTTCCTCCTATGGTACCGTCATTTCTTTCGTCCCATAGGACAAAGGTTT
ACAATCCGAAGACCTTCTTCCCTCACGCGGCGTTGCTGGGTCAGGGTTT
CCCCCATTGCCCAATATTCCCCACTGCTGCCTCCCGTAGGAGTCTGGAC
CGTGTCTCAGTTCCAGTGTGGCCGATCACCCTCTCAGGTCGGCTACCCA
TCGTTGACTTGGTGGGCCGTTACCTCACCAACTATCTAATGGGACGCGA
GCCCATCCTGCATCGAATAAATCCTTTTACCTCAAAACCATGCGGTTTC
GTGGTCTCATGCGGTATTAGCAGTCGTTTCCAACTGTTGTCCCCCGTTG
CAGGGCAGGTTGCTCACGCGTTACTCACCCGTCCGCCACTCGGTATACC
CACAGTTCCTCCCGAAGGATTCACAAAGGGCAACCT

### Example 2. Effects of the C. minuta Strain DSM 32891 in an Animal Model of Depression Induced by Social Stress

Development of the Acute Social Stress Animal Model and Sample Taking

Male C57BL/6 mice that had reached adolescence (postnatal day 32, Charles River, Les Oncins, France) were used for this study. Said mice were kept in controlled conditions of temperature (23° C.), relative humidity (40-50%) and a 12-hour light/darkness cycle and fed with a standard diet (D12450K, Research diet, Brogaarden, Denmark) for 11 weeks (the first week said mice were kept in quarantine in a room prepared to prevent possible zoonosis). The mice were divided randomly into four experimental groups (n=10). Control group (C), stress group (S), group treated with Christensenella minuta DSM 32891 (S+C. min) and group treated with Bifidobacterium breve MF217 (S+B. breve) used for comparison purposes. The mice in the S+C. min group were treated with an oral dose of the bacterial strain according to the invention (1×10⁹ colony-forming units [CFU]) suspended in 10% skimmed milk; the S+B. breve group was treated with a dose of B. breve (1×10⁹ colony-forming units [CFU]) suspended in 10% skimmed milk. The vehicle or placebo (10% skimmed milk) was administered in the same way to both the control group and the stress group. The treatment or placebo was administered for ten weeks. At the end of these ten weeks, the mice were euthanized by cervical dislocation in order to obtain samples, including blood, gut, brain, fecal content and feces.

Acute Social Stress Model

To induce acute social stress, a model of animal social defeat based on the resident-intruder paradigm was used [20]. In this model, one of two animals (the resident) was allowed to establish territoriality in its own cage. Next, the study mice (intruders), in this case C57BL/6 males, were introduced one by one into the cage of the resident mouse. To do this, aggressive CD-1 strain adult (four-week-old) males (Charles River, Les Oncins, France) were used (as resident aggressors), which had previously been isolated and trained to be more aggressive. For four consecutive days, agonistic encounters were carried out (introduction of a naïve mouse into the cage of the resident for ten minutes) in which physical contact between said mice was allowed and in which the intruder mouse suffered a high level of stress (reflected in the production of high levels of corticosterone). The agonistic encounters took place in a neutral room and not in the animal facility in which said mice were usually kept. The experimental mice (intruders) displayed escape or flight behavior, and also defense/submission behavior after suffering the aggression (threat/attack) from the opponent. The criterion employed to define whether an animal had been defeated was the adoption of a specific posture that indicates defeat. Said posture is characterized by an upright submission posture with limp front paws, the head angled upwards and the ears retracted [21].

The control group was not exposed to social defeat; however, all the mice in this group were introduced for ten minutes into a cage exactly the same as those used to carry out the agonistic encounters. For ten minutes, said mice explored the cage without having contact with any opponent.

Before carrying out the agonistic encounters, the animals had fasted for 12 hours. Immediately after the social defeat, the animals were exposed for two hours to food, water, and water with 3% sucrose.

3% Sucrose Preference Test (SPT)

The 3% sucrose preference test was carried out to evaluate the hedonic/anhedonic behavior associated with depressive conduct. Anhedonic behavior (the inability to feel pleasure) is considered one of the clearest symptoms of depression [22]. Different animal studies have shown that depressed animals consume less 3% sucrose water, this being considered anhedonic behavior.

The test consists of depriving the animals of water for 12 hours and then exposing said animals to two options, either water or water with 3% dissolved sucrose. The bottles of sucrose and water were changed during the two-hour test period to ensure that there was no effect related to a place preference. The amount of 3% sucrose ingested during these two hours would indicate hedonic/anhedonic behavior. A lower sucrose ingestion would indicate anhedonia. The preference for sucrose was calculated as the percentage of sucrose ingested in relation to the total amount of liquid consumed, corrected by body weight.

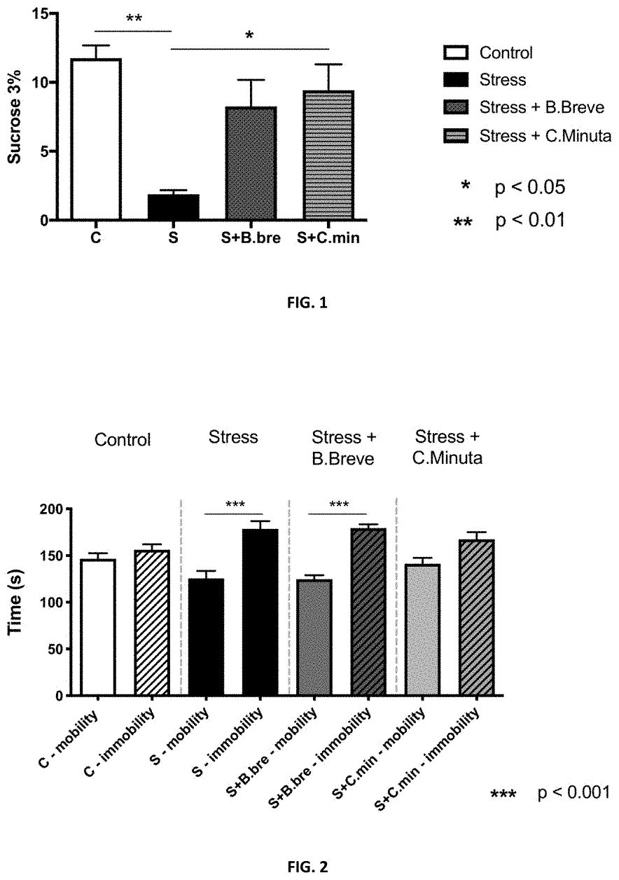

The results (FIG. 1) indicate that the stressed animals (S) ingest significantly less 3% sucrose than the control mice (C) (p<0.01) indicating anhedonic, and therefore depressive, behavior. Treatment of stressed mice with B. breve (S+B. bre) partly remedies anhedonia, although this improvement is not significant. However, treatment of stressed mice with C. minuta (S+C. min) does fully remedy depressive behavior (p<0.05), indicating a greater effectiveness relative to B. breve.

Tail Suspension Test

The mice were suspended from the edge of a table with adhesive tape placed approximately 1 cm from the tip of the tail in a position from which said mice could not escape or hang onto nearby surfaces. Behavior aimed at trying to escape was quantified, as was immobility time for five minutes. The duration of immobility (as a measure of demotivation) was recorded for the five minutes the test lasted. This test is commonly used for evaluating depressive behavior in mice [21].

The results (FIG. 2) indicate that the stressed animals (S) remain immobile for significantly more time relative to the time in which said mice are moving (p<0.001) whereas the control mice (C) show no significant difference. This indicates depressive behavior, as the animals do not try to escape, but give up, showing little motivation to survive. Treatment of stressed mice with B. breve (S+B. bre) does not improve this depressive behavior, with significant differences between immobility time and the time in which the mice were moving being maintained (p<0.001). However, treatment of stressed mice with C. minuta (S+C. min) does remedy this depressive behavior, reducing the time in which the mice are immobile, so that differences between both measurements were reduced and were not significant.

These results demonstrate that oral treatment with C. minuta shows greater efficacy compared with possible conventional probiotics, such as B. breve, in improving depressive behavior in an acute social stress-induced depression model.

The results of both tests demonstrate for the first time that treatment with C. minuta remedies depressive behavior in mice that have been exposed to acute social stress. The data demonstrate that C. minuta would be a better choice as a treatment for improving mood disorders, such as depressive behavior, than conventional probiotics.

### Example 3. Effects of Strain C. minuta DSM 32891 on Stress Markers in an Animal Model of Depression Induced by Social Stress

This example reports the effect of C. minuta DSM 32891 on stress markers in an animal model of social stress and depression (10 days).

For this study, male C57BL/6 mice were used (Charles River, Les Oncins, France). The mice were kept in conditions of controlled temperature (23° C.), relative humidity (40-50%) and light/dark cycle of 12 hours and fed a standard diet (D12450K, Research diet, Brogaarden, Denmark). The mice were randomly divided into 3 experimental groups (n=15/group). The three groups were: a control group (C), stressed group, untreated (S), and stressed group treated with Christensenella minuta DSM 32891 (S+M).

Mice in group S+M were treated daily with an oral dose of the Christensenella minuta DSM 32891 bacterial strain (1×10⁹ colony-forming units (CFU)) suspended in PBS+Glycerol 20%. Mice in groups C and S were treated daily with PBS+Glycerol 20% (placebo).

The treatment of stressed mice with C. minuta or placebo was administered for 38 days. The social defeat protocol (10 days) began after 2 weeks of treatment. The model used is adapted from the resident-intruder paradigm [20], and the time of encounter was reduced to 5 minutes. Adult males of strain CD-1 (4 weeks old) were used as aggressive mice (Charles River, Les Oncins, France), which were previously isolated and trained to be more aggressive. During 10 consecutive days, agonistic encounters (introduction of an aggressive mouse in the resident's cage for 5 minutes) were performed in which physical contact was allowed between them and in which the resident mouse suffered a high degree of stress. The agonistic meetings took place in a neutral room and not in the animal room where they were usually housed. The experimental mice showed evasion or flight behavior, as well as defense/submission behavior after suffering the aggression (threat/attack) on the part of their opponent. The criterion used to define that an animal had been defeated was the adoption of a specific posture that means defeat. It is characterized by a posture of vertical submission with the front legs flaccid, the head tilted upwards and the ears retracted [21].

The control group was not exposed to social defeat; however, all mice in this group were placed for 5 minutes in a cage the same as those used to perform the agonistic encounters. For 5 minutes they explored the cage without having contact with any opponent.

After 38 days, the mice were sacrificed by cervical dislocation and blood samples collected.

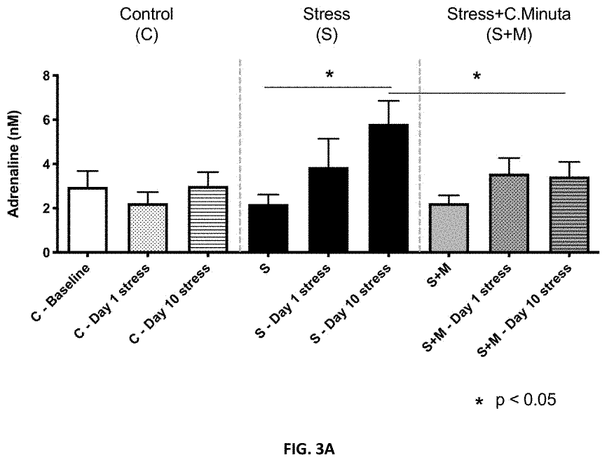

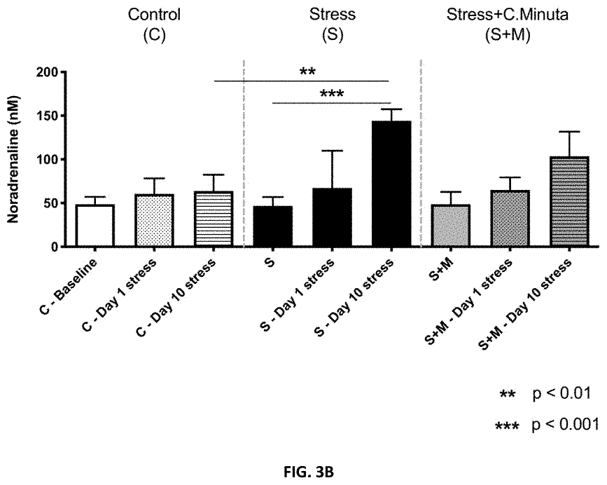

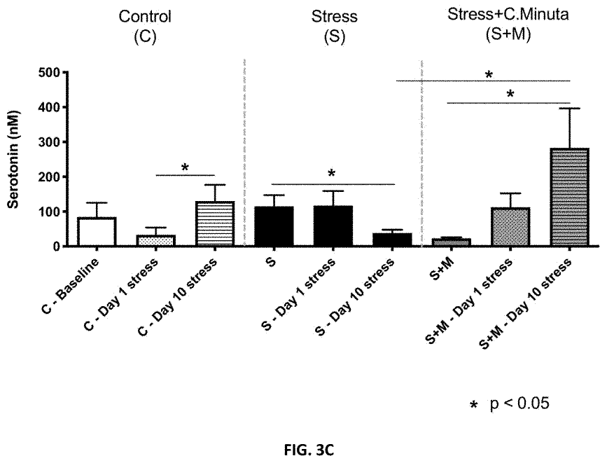

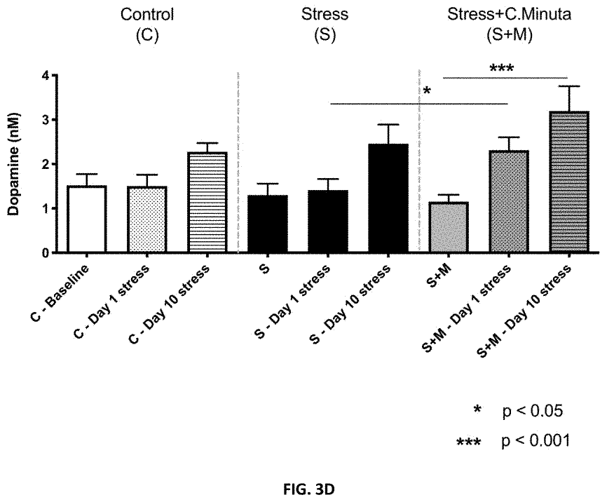

Blood samples were tested for levels of adrenaline, noradrenaline, serotonin, and dopamine at the following time points: at baseline prior to social stress, 4 hours the agonistic encounter on day 1 of social stress, and 4 hours after the agonistic encounter on day 10 of social stress. Results are shown in FIG. 3A-3D; within each experimental group (C: control, S: stressed animals treated with placebo, or S+M: stressed animals treated with C. minuta) the results are shown from left to right for the baseline (before stress), day 1 (after 1 day of social defeat), and day 10 (after 10 days of social defeat) timepoints.

The results indicate that stressed animals (S) exhibited increased levels of adrenaline in the blood 10 days after social defeat (FIG. 3A). Treatment with C. minuta prevented this increase in levels of adrenaline. Similar trends were observed with noradrenaline levels (FIG. 3B).

Additionally, the stressed animals (S) exhibited decreased levels of serotonin in the blood 10 days after social defeat (FIG. 3C). Treatment of stressed mice with C. minuta (S+M) not only prevented this decrease in serotonin levels in stressed mice, but increased serotonin levels 10 days after stress compared to untreated stressed mice. Treatment of stressed mice with C. minuta also significantly increased levels of dopamine in blood (FIG. 3D). This was not observed in control mice nor in untreated stressed mice.

These results demonstrate that treatment with C. minuta was effective to inhibit the effect of stress on the increase of specific markers (adrenaline, noradrenaline), and to increase the levels of serotonin and dopamine in blood during stress.

### Example 4. Sequence Characterization of C. minuta Strain DSM 32891

This example describes comparison of the genomic sequences of C. minuta strain DSM 32891 to the other strains of bacteria belonging to the Christensenellaceae family.

The genome comparison was done using the Genome-to-Genome Distance Calculator v2. 1 Formula 2 [25] to determine a Digital DNA-DNA Hybridization (dDDH) score between each strain and C. minuta strain DSM 32891. If dDDH is higher than 70%, strains belong to the same species. If dDDH is higher than 79%, strains belong to the same species and sub-species. If dDDH is 100%, the strains are identical [26].

| | | #dDDH with strain |
|---|---|---|
| Strain | Strain public reference | DSM 32891 |
| C. minuta | Paper [27] | 96.50% |
| DSM 22607 | Deposited with the DSMZ under | |
| | number DSM 22607 | |
| C. timonensis | Paper [23] | 19.50% |
| DSM 102800 | Deposited with the DSMZ under | |
| | number DSM 102800 | |
| | Other collection N°CSUR P2437 | |
| C. massiliensis | Paper [24] | 21.70% |
| DSM 102344 | Deposited with the DSMZ under | |
| | number DSM 102344 | |
| | Other collection N°CSUR P2438 | |

This analysis, shown in the table above, demonstrates that strain DSM 32891 has a dDDH lower than 70% with strains C. timonensis and C. massiliensis, confirming that they belong to different species. This is in agreement with the fact that DSM 32891 belongs to Christensenella minuta species.

This also shows that strain DSM 32891 and strain DSM 22607 have a dDDH of 96.5%, higher than 79%, and belongs to the same sub-species, but are different strains.

This confirms that strain DSM 32891 is a novel strain belonging to the species Christensenella minuta.

### BIBLIOGRAPHY

1. Gustavsson, A., et al., Cost of disorders of the brain in Europe 2010. Eur Neuropsychopharmacol, 2011. 21(10): p. 718-79.
2. Lamers, F., et al., Evidence for a differential role of HPA-axis function, inflammation and metabolic syndrome in melancholic versus atypical depression. Mol Psychiatry, 2013. 18(6): p. 692-9.
3. Milaneschi Y, L. F., Bot M, Drent M L, Penninx B W, Leptin Dysregulation Is Specifically Associated With Major Depression With Atypical Features: Evidence for a Mechanism Connecting Obesity and Depression. Biol Psychiatry., 2017. May 1; 81(9): p. 807-814.
4. Zhernakova, A., et al., Population-based metagenomics analysis reveals markers for gut microbiome composition and diversity. Science, 2016. 352(6285): p. 565-9.
5. McDonald, D., et al., American Gut: an Open Platform for Citizen Science Microbiome Research. mSystems, 2018. 3(3).
6. Aizawa, E., et al., Possible association of Bifidobacterium and Lactobacillus in the gut microbiota of patients with major depressive disorder. J Affect Disord, 2016. 202: p. 254-7.
7. Jiang, H., et al., Altered fecal microbiota composition in patients with major depressive disorder. Brain Behav Immun, 2015. 48: p. 186-94.
8. Kelly, J. R., et al., Transferring the blues: Depression-associated gut microbiota induces neurobehavioural changes in the rat. J Psychiatr Res, 2016. 82: p. 109-18.
9. Naseribafrouei A, H. K., Avershina E, Sekelja M, Linløkken A, Wilson R, Rudi K., Correlation between the human fecal microbiota and depression. Neurogastroenterol Motil., 2014. August; 26(8): p. 1155-62.
10. Gibson-Smith, D., et al., The role of obesity measures in the development and persistence of major depressive disorder. J Affect Disord, 2016. 198: p. 222-9.
11. De Palma, G., et al., Microbiota and host determinants of behavioural phenotype in maternally separated mice. Nat Commun, 2015. 6: p. 7735.
12. Sanz, Y., Bifidobacteria in foods: health effects, in The Encyclopedia of Food and Health.B. Caballero, P. Finglas, and F. Toldrá, Editors. Oxford Academic Press: Oxford., 2016: p. p. 388-394.
13. Dinan, T. G. and J. F. Cryan, Mood by microbe: towards clinical translation. Genome Med, 2016. 8(1): p. 36.
14. Romijn, A. R., et al., A double-blind, randomized, placebo-controlled trial of Lactobacillus helveticus and Bifidobacterium longum for the symptoms of depression. Aust N Z J Psychiatry, 2017. 51(8): p. 810-821.
15. Pinto-Sanchez, M. I., et al., Probiotic Bifidobacterium longum NCC3001 Reduces Depression Scores and Alters Brain Activity: A Pilot Study in Patients With Irritable Bowel Syndrome. Gastroenterology, 2017. 153(2): p. 448-459 e8.
16. Yu, M., et al., Variations in gut microbiota and fecal metabolic phenotype associated with depression by 16S rRNA gene sequencing and LC/MS-based metabolomics. J Pharm Biomed Anal, 2017. 138: p. 231-239.
17. I. Mironova, I. Z., N. Zhukova, V. Alifirova, A. Latypova, O. Izhboldina, M. Nikitina, V. Petrov, M. Titova, Affective disorders and microbiome in patients with Parkinson's disease (PR2063). European Journal of Neurology., 2017. 24 (Suppl. 1): p. 445-678.
18. Gibson G R, C. J., Macfarlane G T., Use of a three-stage continuous culture system to study the effect of mucin on dissimilatory sulfate reduction and methanogenesis by mixed populations of human gut bacteria. Appl Environ Microbiol. 1988 November; 54(11), 1988. November; 54(11): p. 2750-5.
19. Lesmes, U., et al., Effects of resistant starch type Ill polymorphs on human colon microbiota and short chain fatty acids in human gut models. J Agric Food Chem, 2008. 56(13): p. 5415-21.
20. Tamashiro, K. L., M. M. Nguyen, and R. R. Sakai, Social stress: from rodents to primates. Front Neuroendocrinol, 2005. 26(1): p. 27-40.
21. Can, A., et al., The tail suspension test. J Vis Exp, 2012(59): p. e3769.
22. Duncko, R., et al., Altered function of peripheral organ systems in rats exposed to chronic mild stress model of depression. Cell Mol Neurobiol, 2001. 21(4): p. 403-11.
23. Ngondo, S, et al., Christensenella timonensis, a new bacterial species isolated from the human gut. New Microbe and New Infect 2016; 13: 32-33.
24. Ngondo, S, et al., Christensenella massiliensis, a new bacterial species isolated from the human gut. New Microbe and New Infect 2016; 12: 69-70.
25. Meir Kolthof J P, et al., Genome sequence-based species delimitation with confidence intervals and improved distance functions, BMC Bioinformatics volume 14, Article number: 60 (2013).
26 Meir KolthofJP, et al., Complete genome sequence of DSM 30083^{T}, the type strain (U5/41^{T}) of Escherichia coli, and a proposalfor delineating subspecies in microbial taxonomy, Standards in Genomic Sciences volume 9, Article number: 2 (2014).
27 Morotomy M, et al., Description of Christensenella minuta gen. nov., sp. nov., isolated from human faeces, which forms a distinct branch in the order Clostridiales, and proposal of Christensenellaceae fam. nov., International Journal of Systematic and Evolutionary Microbiology (2012), 62, 144-149.

1. A strain of Christensenella minuta with the deposit number DSM 32891.

2. A strain derived from the strain according to claim 1.

3. The strain according to claim 1 in which the strain is a genetically modified mutant.

4. The strain according to claim 1, wherein said strain is in the form of a viable or a non-viable cell.

5. A cellular component, metabolite, secreted molecule or any combination thereof obtained from the strain according to claim 1.

6. A composition which comprises a strain of Christensenella minuta according to claim 1 or a cellular component, metabolite, secreted molecule or any combination thereof obtained from the strain.

7. The composition according to claim 6, wherein the composition also comprises at least one bioactive component.

8. The composition according to claim 6, wherein the composition also comprises at least one microorganism that is of a different strain than the bacterium according to claim 1.

9. The composition according to claim 8, wherein the microorganism is a gut bacterium or a lactic bacterium.

10. The composition according to claim 6, wherein said composition is a pharmaceutical composition.

11. The composition according to claim 10, wherein the composition also comprises at least one pharmaceutically acceptable vehicle and/or excipient.

12. The composition according to claim 10, wherein said composition is presented in a form suitable for oral, sublingual, nasal, bronchial, lymphatic, rectal, transdermal, inhaled or parenteral administration.

13. The composition according to claim 6, wherein said composition is a nutritional composition.

14. The composition according to claim 13, wherein the nutritional composition is a foodstuff, a supplement, a nutraceutical, a probiotic or a symbiotic.

15. The composition according to claim 14, wherein said foodstuff is selected from the list that is made up of a milk product, plant product, meat product, aperitif, chocolate, drink or baby food.

16. The composition according to claim 6, wherein said composition has a concentration of the bacterium of between 10⁴ and 10¹⁴ colony-forming units (CFU) per gram or milliliter of final composition.

17. The strain of Christensenella minuta according to claim 1, a cellular component, metabolite, secreted molecule or any combination thereof obtained from the strain, or a composition containing the strain or the cellular component, metabolite, secreted molecule or any combination thereof, for use as a medicament or as a medicine.

18. A Christensenellaceae bacterium or a composition comprising a Christensenellaceae bacterium, for use in the prevention and/or treatment of a mood disorder and/or stress disorder and/or anxiety disorder and/or migraine.

19. A Christensenellaceae bacterium or a composition comprising a Christensenellaceae bacterium, for use as an adjuvant in treatments for mood disorders and/or stress disorders and/or anxiety disorders and/or migraines.

20. The Christensenellaceae bacterium or a composition comprising a Christensenellaceae bacterium for use according to claim 18, wherein the mood disorders are selected from the list which comprises: depression, major depression, atypical depression, typical or melancholic depression, psychotic depression, catatonic depression, pre- and post-partum depression, bipolar disorder, seasonal affective disorder, dysthymia, depressive personality disorder, double depression, unspecified depressive disorder, recurrent brief depressive disorder, minor depression, alterations of the state of mind, and mood disorder induced by substance abuse or by the use of drugs, such as drugs of abuse.

21. The Christensenellaceae bacterium for use according to claim 18, wherein said stress disorder is selected from reactive attachment disorder, disinhibited social engagement disorder, posttraumatic stress disorder (PTSD), acute stress disorder, and adjustment disorders.

22. The Christensenellaceae bacterium for use according to claim 18, wherein said anxiety disorder is selected from panic attack, panic disorder, agoraphobia, social phobia or social anxiety disorder, obsessive-compulsive disorder, post-traumatic stress disorder (ASD), generalized anxiety disorder, or acute stress disorder.

23. The Christensenellaceae bacterium or a composition comprising a Christensenellaceae bacterium for use according to claim 18, wherein said Christensenellaceae bacteria are bacteria of the genus Christensenella.

24. The Christensenellaceae bacterium or a composition comprising a Christensenellaceae bacterium for use according to claim 23, wherein said Christensenella bacteria are selected from the species Christensenella minuta, Christensenella timonensis, Christensenella massiliensis or any of the combinations thereof.

25. The Christensenellaceae bacterium or a composition comprising a Christensenellaceae bacterium for use according to claim 24, wherein said Christensenella bacteria is a Christensenella massiliensis strain with the deposit number DSM 102344 or a Christensenella timonensis strain with the deposit number DSM 102800 any of the combinations thereof.

26. The Christensenellaceae bacterium or a composition comprising a Christensenellaceae bacterium for use according to claim 24, wherein said Christensenella bacterium is a strain of Christensenella minuta with the deposit number DSM 32891, a strain of Christensenella minuta with the deposit number DSM 22607 or any of the combinations thereof.

27. The Christensenellaceae bacterium or a composition comprising a Christensenellaceae bacterium for use according to claim 18, wherein said Christensenellaceae bacteria are in the form of viable cells and/or in the form of non-viable cells.

28. The Christensenellaceae bacterium or a composition comprising a Christensenellaceae bacterium for use according to claim 18, wherein said Christensenellaceae bacterium comprises a 16S rRNA sequence having at least 90% identity, at least 95% identity, at least 96% identity, at least 97% identity, at least 98% identity, at least 99% identity, or at least 99.5% identity to the 16S rRNA sequence of the strain of Christensenella minuta with the deposit number DSM 32891 (SEQ ID NO: 3), or wherein said Christensenellaceae bacterium comprises the strain of Christensenella minuta with the deposit number DSM 32891.

29. The bacterium for use according to claim 17, which is for use in a subject selected from a human or a non-human animal.

30. The bacterium for use according to claim 29, wherein said non-human animal is selected from companion animals, livestock animals, zoo animals, mammals, dogs, cats, guinea pigs, ferrets, hamsters, pigs, cows, goats, sheep, horses, mice, and rats.

31. The bacterium for use according to claim 17, wherein said use comprises administration of between 10⁴ and 10¹⁴ colony-forming units (CFU) of said bacteria.

32. Use of the strain according to claim 1, a cellular component, metabolite, secreted molecule or any of its combinations obtained from the strain, or a composition comprising the strain, the cellular component, metabolite, secreted molecule or any of its combinations, for the preparation of a food.
